(19) United States
(12) Patent Application Publication
(10) Pub. No.: US 2011/0207972 A1
(43) Pub. Date: Aug. 25, 2011

(54) CATALYSTS AND PROCESSES FOR THE HYDROGENOLYSIS OF GLYCEROL AND OTHER ORGANIC COMPOUNDS FOR PRODUCING POLYOLS AND PROPYLENE GLYCOL

(75) Inventors: Heather M. Brown, Richland, WA (US); John G. Frye, Richland, WA (US); Jonathan L. Male, Richland, WA (US); Daniel M. Santosa, Richland, WA (US); Alan H. Zacher, Richland, WA (US)

(73) Assignee: Battelle Memorial Institute

(21) Appl. No.: 12/711,020

(22) Filed: Feb. 23, 2010

(51) Int. Cl. C07C 31/20; B01J 23/10; B01J 23/83; B01J 23/883; B01J 23/889; B01J 23/44; B01J 23/50; B01J 23/755; B01J 21/18; B01J 23/75; B01J 23/882

(52) U.S. Cl. 568/861; 502/303; 502/302; 502/304; 502/315; 502/324; 502/326; 502/330; 502/337; 502/184; 502/185; 502/325; 502/313

(57) ABSTRACT

Catalysts for replacing rhenium-containing multimetallic catalysts for the hydrogenolysis of organic compounds to desired polyols, including the conversion of glycerol to propylene glycol, are described. The catalysts are carried on carbon supports, as well as carbon supports impregnated with Zirconium Scandium (ZrSc), Zirconium Yttrium (ZrY), Titanium Scandium (TiSc), or Titanium Yttrium (TiY) to texture the carbon support and to create oxygen-ion vacancies that can be used during the desired reactions. Processes for the hydrogenolysis of organic compounds to desired polyols using the disclosed catalysts, including the conversion of glycerol to propylene glycol, are also described.

### ACKNOWLEDGMENT OF GOVERNMENT SUPPORT

This invention was made with Government support under Contract DE-AC06-76RLO1830 awarded by the U.S. Department of Energy. The Government has certain rights in the invention.

### FIELD

This disclosure relates to rhenium-free catalysts for hydrogenolysis processes including the conversion of glycerol to polyols such as propylene glycol, and processes for using the same.

### BACKGROUND

The hydrogenolysis of certain organic compounds for the production of selected polyols, such as the conversion of glycerol to propylene glycol, is facilitated by catalysts having group eight metals supported on carbon. Bi-metallic and tri-metallic catalyst compositions have proved optimal for the conversion of glycerol to propylene glycol because they allow for the balancing between the several opposing reactions during the conversion process. As disclosed in U.S. Pat. No. 6,841,085, for example, exemplary catalysts found thus far include rhenium-containing multimetallic catalysts, such as bi-metallic nickel/rhenium compositions and tri-metallic cobalt/palladium/rhenium compositions on a carbon support surface.

The common element in these compositions is rhenium. It is thought that the rhenium component performs three functions during the hydrogenolysis of organic compounds. First, the rhenium component appears to be highly dispersed across the entire carbon support surface, thus functioning as a textural promoter while also helping to maintain the other metals in a highly dispersed state. Second, some portion of the rhenium appears to be alloyed with either the Ni or Co and may be altering the reactivity of those metals through electronic interactions. Finally, it is thought that the rhenium is in a partially reduced state and provides oxygen acceptor sites that facilitate removal of hydroxyl groups from intermediate species during the reaction sequence. This may also account for the strong interaction with the carbon support surface (via interaction with oxygen-containing functional groups found on the carbon support surface).

Although such rhenium-containing catalysts are effective for the hydrogenolysis of at least certain organic compounds for the production of selected polyols, including the conversion of glycerol to propylene glycol, one drawback to rhenium is that it is costly and thus is less likely to be utilized in an industrial setting. Thus, there is a need for effective and less costly catalysts for the conversion of glycerol to propylene glycol and for other hydrogenolysis processes.)

In addition, when rhenium containing catalysts are used in aqueous phase applications, care must be taken to maintain the catalyst under reducing conditions while the catalyst is in contact with water. If rhenium becomes oxidized, its water solubility is likely to increase and it can be leached from the catalyst more readily. Rhenium tends to form anionic complexes, which generally have high water solubility. Compounds, such as perrhenic acid (HReO₄), are frequently used as water soluble rhenium precursors when fabricating such catalysts. Thus, there is a need for lower water soluble compositions to prevent such undesirable leaching and breakdown of the catalyst.

### SUMMARY

Disclosed herein are rhenium free catalysts for facilitating the hydrogenolysis of certain organic compounds for the production of selected polyols, including the conversion of glycerol to polyols such as propylene glycol. Processes for using such catalysts are also disclosed.

The disclosed multimetallic catalysts of certain embodiments include nickel and at least one of La, Sm, Ce, Ru, Ag, Pr, Mn, Co, Pd, Cr, Mo, Zr, and Fe. The disclosed catalysts facilitate the hydrogenolysis of organic compounds including the conversion of glycerol to propylene glycol. In other embodiments, the disclosed multimetallic catalysts include cobalt and at least one of Ni, Ir, Mo or Ce. In certain examples, the disclosed catalysts include nickel/lanthanum catalysts, and nickel/praseodymium/cerium catalysts.

The disclosed catalysts include carbon supports. In some embodiments, the carbon support is an acid washed extruded carbon support. The carbon supports of the disclosed catalysts can be modified with Zirconium Scandium (ZrSc), Zirconium Yttrium (ZrY), Titanium Scandium (TiSc), or Titanium Yttrium (TiY) to texture the carbon support and to create oxygen-ion vacancies that can be used during the desired reactions.

Also disclosed in connection with the hydrogenolysis of organic compounds are compositions of a multimetallic catalyst comprising nickel and at least one of La, Sm, Ce, Ru, Ag, Pr, Mn, Co, Pd, Cr, Mo, Zr, and Fe, along with hydrogen and at least one of a carbon sugar, a carbon sugar alcohol, or glycerol. In other embodiments, disclosed in connection with the hydrogenolysis of organic compounds are compositions of a multimetallic catalyst comprising cobalt and at least one of Ni, Ir, Mo or Ce, along with hydrogen and at least one of a carbon sugar, a carbon sugar alcohol, or glycerol.

Also disclosed herein are hydrogenolysis processes or methods that involve reacting a composition comprising a carbon sugar, a carbon sugar alcohol, or glycerol with hydrogen in the presence of a solid multimetallic catalyst comprising nickel and at least one of La, Sm, Ce, Ru, Ag, Pr, Mn, Co, Pd, Cr, Mo, Zr, and Fe.

Also disclosed herein are processes for making propylene glycol that involve reacting a composition comprising glycerol with hydrogen in the presence of a solid multimetallic catalyst comprising nickel and at least one La, Sm, Ce, Ru, Ag, Pr, Mn, Co, Pd, Cr, Mo, Zr, and Fe. In another embodiment, the process involves reacting a composition comprising glycerol with hydrogen in the presence of a solid multimetallic catalyst comprising cobalt and at least one of Ni, Ir, Mo or Ce. The processes disclosed herein achieve selectivity of propylene glycol of about 50% and greater.

The foregoing and other objects, features, and advantages of the invention will become more apparent from the following detailed description.

### BRIEF DESCRIPTION OF THE DRAWINGS

FIG. 1 is a graph illustrating improved catalyst activity with modified supports.

FIG. 2 is a graph showing combinatorial plate results of novel metal matrices.

### DETAILED DESCRIPTION

Disclosed herein are catalyst compositions for the hydrogenolysis of organic compounds for the production of selected polyols, including the conversion of glycerol to polyols, such as propylene glycol. A set of catalysts are disclosed that can convert glycerol in high yields without the presence of rhenium, thus reducing manufacturing costs.

Also disclosed are systems and processes for using the catalysts to facilitate the conversion of glycerol to polyols, including propylene glycol, as well as the hydrogenolysis of other organic compounds to desired products.

The disclosed catalysts are rhenium-free but maintain the functionality and/or achieve comparable results of rhenium-containing catalysts. In some embodiments, the metals have significantly different properties than rhenium. For instance, the disclosed compositions tend to form cationic species whose oxides and hydroxides have low water solubility under neutral to basic pH conditions. Loss of reducing conditions for most of these elements is not expected to result in the loss of these metals from the catalysts.

In other cases, modification of the carbon support eliminated the need for the rhenium component. In any event, the disclosed catalysts achieved surprisingly superior results. In addition, and importantly, the disclosed catalysts provide a significant commercial advantage as a result of their overall lower costs and effectiveness as compared to rhenium-containing catalysts.

The catalysts were prepared and tested using a high-throughput batch screening system. Those which gave glycerol conversions about 50% or greater were chosen as suitable substitutes for rhenium-containing catalysts. Specifically, the target systems to meet or exceed consisted of a 5% Nickel (Ni)/1% Rhenium (Re) on Norit® ROX 0.8, and a 2.5% Cobalt (Co)/0.45% Palladium (Pd)/2.37% Re on Norit® ROX 0.8. (Norit® ROX 0.8 mm is an acid washed extruded carbon available from Norit® Americas, Inc. (Marshall, Tex.)). These catalysts had average glycerol conversions of 68.0±0.2% and 56.6±2.4% respectively.

The disclosed catalyst compositions are shown in Table 1 and Table 2. They are split between metals supported on unmodified Norit® ROX 0.8 (Manufacturer Lot. No. 570393), and Norit® ROX 0.8 which has been modified by impregnation with Zirconium Scandium (ZrSc), Zirconium Yttrium (ZrY), Titanium Scandium (TiSc), or Titanium Yttrium (TiY) before additional metals were added.

By way of example, the recipe for preparing an approximately 40 g batch of a 5% Ni/0.251% Pr/2.249% Ce Norit ROX 0.8 catalyst (Lot No. 570393) is provided immediately below.

Metals impregnation solution preparation:
1) add 10.7151 g of Ni(NO₃)₂.6H₂O to a 60 cc plastic centrifuge tube;
2) add 3.8061 g of (NH₄)₂Ce(NO₃)₆;
3) add 0.3446 g of Pr(NO₃)₃.6H₂O;
4) add DI water and dilute up to total volume of 42 ml; and
5) swirl gently until dissolved.

Carbon support impregnation:
1) add 40.91 g of (Lot No. 570393) Norit ROX 0.8 mm extrudate to a 16 oz wide-mouth jar;
2) secure the jar into the jaw of a rotary coating unit designed to tumble the support at approximately a 45 degree angle, speed set to 2.5 (approximately 60 rpm);
3) ensure the impregnation solution is fully dissolved, then slowly add drop wise to the tumbling support in the open jar;
4) place the cap on the jar and allow to tumble for approximately 1 hour;
5) remove cap and aim a laboratory heat gun towards the tumbling catalyst (gently enough to not blow catalyst out of the jar, and continue until dry which is determined by periodic checks with a cooled (below room temperature) watch glass placed over the mouth of the jar until no condensation is detected);
6) transfer the jar to a vacuum oven at 60° C. under house vacuum overnight;
7) removed the jar and empty into a catalyst storage vial; and
8) cap, cool, and reduce before use.

The catalysts generally have Ni in a weight percent from about 2% to about 7%, and preferably about 5%. The catalysts were tested in a batch mode, with approximately 35 mg catalyst, 150 μL of a 10% glycerol/1% sodium hydroxide feed solution, at 1400 psig H₂, 700 rpm stirring, and a 4 hour run time. Each catalyst was reduced prior to reaction by heating to 320° C. at 1.5° C./min and holding for 6 hours under 100 mL/min H₂ flow.

TABLE 1

| | | | | | | |
|---|---|---|---|---|---|---|
| | Catalyst | Glycerol | PG | EG | Molar | Mass |
| Catalyst | Metal Loading | Conversion | Selectivity | Selectivity | Balance | Recovery |
| Number | (wt %) | (STDEV) | (STDEV) | (STDEV) | (STDEV) | (STDEV) |

Top Performing Ni containing catalyst compositions on Norit ® ROX

| | | | | | | | | | | | |
|---|---|---|---|---|---|---|---|---|---|---|---|
| A3 | 5Ni1Ru | 56.3% | 0.8% | 52.1% | 0.6% | 7.2% | 0.1% | 85.6% | 0.9% | 27.0% | 0.9% |
| A4 | 5Ni0.81Sm | 51.1% | 1.7% | 54.4% | 0.9% | 5.8% | 0.2% | 84.4% | 0.2% | 27.5% | 0.8% |
| A5 | 5Ni0.75La | 57.7% | 0.4% | 55.7% | 1.2% | 5.3% | 0.2% | 82.8% | 0.9% | 26.8% | 0.5% |
| A6 | 5Ni0.75Ce | 50.1% | 1.0% | 53.9% | 2.8% | 5.4% | 0.3% | 83.7% | 1.3% | 27.1% | 0.7% |
| A7 | 5Ni0.54Pr0.22Ce | 51.8% | 1.7% | 55.1% | 0.8% | 5.6% | 0.3% | 84.0% | 0.5% | 25.9% | 1.1% |
| A14 | 5Ni0.25Ag0.43Ce | 51.5% | 1.4% | 53.6% | 1.2% | 5.4% | 0.2% | 83.6% | 0.8% | 26.6% | 0.4% |
| A19 | 10Ni | 53.3% | 1.1% | 57.0% | 1.2% | 7.4% | 0.2% | 85.8% | 0.7% | 26.4% | 0.4% |
| A62 | 5Ni 0.54Pr 0.22Ce | 50.9% | 1.8% | 61.8% | 0.9% | 4.5% | 0.2% | 85.9% | 0.3% | 93.8% | 0.1% |
| A95 | 5Ni1Ru | 61.1% | 1.5% | 57.4% | 0.8% | 7.9% | 0.3% | 88.8% | 0.5% | 92.2% | 0.1% |
| A96 | 5Ni0.81Sm | 66.3% | 2.6% | 68.5% | 1.2% | 5.9% | 0.1% | 87.9% | 0.3% | 92.4% | 0.1% |
| A97 | 5Ni0.75La | 71.7% | 1.4% | 67.6% | 0.8% | 5.6% | 0.2% | 85.8% | 0.4% | 92.1% | 0.0% |
| A98 | 5Ni0.75Ce | 63.7% | 2.1% | 67.0% | 1.2% | 5.4% | 0.1% | 86.6% | 0.3% | 91.7% | 0.3% |
| A99 | 5Ni0.54Pr0.22Ce | 56.5% | 3.6% | 68.5% | 1.0% | 5.5% | 0.0% | 88.6% | 0.1% | 92.3% | 0.2% |
| A101 | 5Ni0.3Mn | 53.9% | 1.5% | 69.3% | 1.7% | 5.5% | 0.1% | 90.0% | 0.5% | 92.4% | 0.1% |
| A103 | 5Ni0.32Co | 51.5% | 1.3% | 67.8% | 1.7% | 5.4% | 0.2% | 89.2% | 0.6% | 92.1% | 0.1% |
| A106 | 5Ni0.25Ag0.43Ce | 57.5% | 1.8% | 48.3% | 41.9% | 4.1% | 3.6% | 75.3% | 28.2% | 92.3% | 0.1% |
| A108 | 5Ni0.25Ag0.17Mn | 51.5% | 1.3% | 69.3% | 1.6% | 5.5% | 0.2% | 90.4% | 0.7% | 92.3% | 0.1% |
| A111 | 10Ni | 54.4% | 4.4% | 66.0% | 0.8% | 6.8% | 0.3% | 89.3% | 0.4% | 91.9% | 0.1% |

Top performing Co containing catalyst compositions on Norit ® ROX

| | | | | | | | | | | | |
|---|---|---|---|---|---|---|---|---|---|---|---|
| A11 | 5Ni0.32Co | 46.4% | 0.5% | 51.7% | 0.8% | 5.2% | 0.1% | 84.0% | 0.1% | 26.9% | 0.5% |
| A27 | 2.5Co 2.45Ir 0.41Mo | 43.5% | 1.5% | 51.3% | 0.9% | 4.1% | 0.6% | 85.4% | 5.1% | 100.5% | 0.0% |
| A30 | 2.5Co 2.37Re | 43.7% | 2.0% | 67.9% | 0.8% | 4.9% | 0.6% | 91.6% | 3.1% | 100.5% | 0.0% |
| A38 | 2.5Co 0.81Ir 0.7Mn | 41.7% | 1.9% | 51.8% | 1.6% | 4.8% | 0.2% | 87.5% | 1.2% | 100.3% | 0.1% |

TABLE 2
Catalyst compositions on modified Norit ® ROX

| | Catalyst | Glycerol | | PG | | EG | | Molar | | Mass | |
|---|---|---|---|---|---|---|---|---|---|---|---|
| Catalyst | Metal Loading | Conversion | | Selectivity | | Selectivity | | Balance | | Recovery | |
| Number | (wt %) | (STDEV) | | (STDEV) | | (STDEV) | | (STDEV) | | (STDEV) | |
| A113 | 5Ni 0.75La | 66.4% | 0.1% | 65.4% | 0.4% | 5.6% | 0.3% | 86.4% | 0.3% | 95.2% | 0.1% |
| A114 | 5Ni 0.59Mn | 61.3% | 6.6% | 68.9% | 6.9% | 5.2% | 1.6% | 91.4% | 6.4% | 95.0% | 0.0% |
| A116 | 5Ni 0.45Pd | 63.5% | 0.5% | 61.8% | 0.1% | 5.9% | 0.1% | 86.7% | 0.3% | 94.9% | 0.2% |
| A117 | 5Ni 0.28Cr | 57.1% | 1.6% | 65.5% | 0.2% | 5.1% | 0.1% | 88.1% | 0.3% | 95.3% | 0.2% |
| A118 | 5Ni | 58.3% | 1.4% | 67.0% | 5.4% | 5.3% | 0.5% | 89.5% | 3.8% | 95.0% | 0.0% |
| A126 | 0.32Co 5Ni | 59.6% | 0.4% | 65.2% | 0.2% | 5.1% | 0.0% | 88.0% | 0.2% | 95.1% | 0.0% |
| A128 | 5Ni 0.75La | 64.6% | 1.4% | 64.1% | 0.8% | 5.1% | 0.1% | 85.7% | 0.3% | 95.1% | 0.1% |
| A129 | 5Ni 0.59Mn | 57.8% | 0.2% | 63.2% | 0.2% | 4.8% | 0.3% | 87.0% | 0.9% | 95.0% | 0.1% |
| A131 | 5Ni 0.45Pd | 61.6% | 1.6% | 65.0% | 6.7% | 5.7% | 0.7% | 89.1% | 5.9% | 95.1% | 0.1% |
| A132 | 5Ni 0.28Cr | 54.9% | 1.4% | 64.2% | 0.7% | 4.8% | 0.1% | 87.4% | 0.4% | 95.2% | 0.1% |
| A133 | 5Ni | 56.8% | 2.1% | 62.0% | 0.8% | 4.6% | 0.0% | 86.1% | 0.6% | 94.7% | 0.1% |
| A140 | 0.32Co 5Ni | 58.5% | 1.1% | 61.6% | 1.3% | 4.7% | 0.1% | 86.1% | 0.8% | 95.0% | 0.3% |
| A145 | 5Ni 0.45Pd | 51.8% | 0.9% | 65.4% | 3.4% | 5.3% | 0.2% | 89.5% | 1.8% | 97.8% | 0.1% |
| A160 | 5Ni 0.45Pd | 50.5% | 1.9% | 64.2% | 1.1% | 5.1% | 0.2% | 88.2% | 0.3% | 109.6% | 0.2% |

Note:
A113-A126 = 2.89% Zr/0.11% Sc on Norit ® Rox 0.8
A128-A140 = 2.78% Zr/0.22% Y on Norit ® Rox 0.8
A145 = 2.79% Ti/0.21% Sc on Norit ® Rox 0.8
A160 = 2.61% Ti/0.39% Y on Norit ® Rox 0.8

As set forth in Table 1, the 5% Ni/0.75% Lanthanum (La) on Norit® ROX 0.8 catalyst gave the highest glycerol conversion of 71.7% with a propylene glycol selectivity of 67.6%.

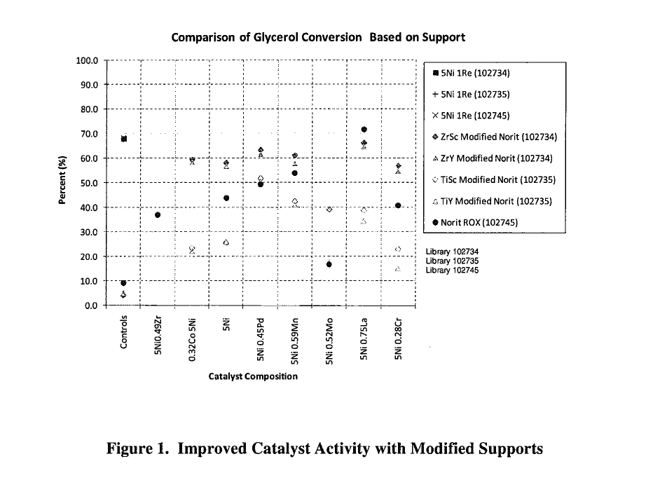

The catalyst testing results indicate that textural promoters are definitely a valid way to replace rhenium in active catalyst compositions. They provide many of the same functions as rhenium, are often less expensive, and can give similar catalyst activities. In particular, many of the compositions on Norit® ROX 0.8 showed improved glycerol conversion when they were added to a support pre-impregnated with ZrSc, ZrY, TiSc, or TiY. A graphical comparison with respect to some of the catalyst disclosed herein on such modified supports can be seen in FIG. 1.

Modification of the Norit® Rox 0.8 support provided interesting results. In many cases, glycerol conversion was improved up to 10% when facilitated by catalysts having modified supports compared to those without modified supports. It is believed that the improved conversion is due to a texturing effect produced when the support is first impregnated with a group IV metal, doped with a group III metal. The texturing allows for better dispersion of the group eight metal across the support, and the doping of the zirconium or titanium creates oxygen-ion vacancies that can be used during the reaction. The discovered modified supports may have even wider application to other metal matrices for this chemistry.

It is interesting to note that even though zirconium and titanium are both from group IV of the periodic table, the catalysts made on the titanium modified Norit ROX 0.8 support did not show the improved conversion that the catalysts made on the zirconium modified Norit ROX 0.8 support showed. This is likely due to the difference in the way that the two modified supports were made. In the case of the zirconium modified carbon supports, the zirconium, scandium and yttrium were all added as aqueous nitrate solutions to the carbon support. The solutions, while added separately to the support, were mixed together on the carbon support prior to drying and calcining. This results in a more homogeneous mixture of the zirconium and dopant (either scandium or yttrium), and upon calcination likely results in a mixed oxide structure containing oxygen ion vacancies, thought to play an important role in the subsequent reactions. The titanium modified carbon supports were made using an alcoholic solution of titanium (IV) isopropoxide as the titanium precursor. The carbon support in this case was impregnated with the titanium isopropoxide, hydrolyzed with water, then dried, likely resulting in a uniformly cross-linked titanium coating. The dopants (scandium and yttrium) were then added as aqueous nitrate solutions to the titanium-coated carbon supports, and subsequently dried and calcined. It is less likely that the Sc and Y dopants added in this manner are as readily incorporated into the titanium oxide layer, resulting in a lower population of oxygen ion vacancies. It would be expected that if the titanium modification would have been conducted in one step, using a mixture of titanium (IV) isopropoxide and either scandium (III) or yttrium (III) isopropoxide, that a more homogeneous mixed oxide phase would likely have resulted, and the conversions in subsequent testing improved.

The Ni/La composition appears to rely on the textural improvement of lanthanum alone; however, it is unusual that nearly a 50% improvement in the catalyst activity of Ni alone was seen with Ni/La, while practically no improvement was seen with Ni/Zr, which should act similarly to Ni/La. See FIG. 1.

The disclosed compositions have the potential to reduce catalyst manufacturing costs by replacing previously known standard catalysts. In addition, the catalysts could also be applied to the hydrogenolysis of xylitol and sorbitol feed stocks, or even to simple sugars, such as glucose, fructose, or xylose. The disclosed catalyst compositions could possibly replace chromium-containing catalysts, such as copper chromite catalysts. Copper chromite catalysts have routinely been used in the conversion of esters to alcohols. It is also well known that a portion of the chromium contained in commercial copper chromite catalysts is present as chromium+6, a known carcinogen.

The novel catalysts disclosed above were generated by combinatorial testing. The Nickel/Praseodymium/Cerium (Ni/Pr/Ce) catalyst on a modified carbon support was tested in a trickle bed flow reactor. The catalyst was tested against both glycerol and xylitol to examine the effect of the catalyst for hydrogenolysis of various polyols (See Table 4.) Catalyst loading and reduction conditions for the modified Ni/Pr/Cr catalyst tested in the flow reactor are presented in Table 3.

TABLE 3
Catalyst Information

| | | | | | | Reduction Details | | |
|---|---|---|---|---|---|---|---|---|
| | | | | Cal vol | | Ramp Rate | T(° C.)/ | % H2 in |
| Run # | Catalyst Composition | Cat LRB# | Reactor | (cc) | Wt(g) | (° C./min) | Duration | inert gas |
| F179 | 5% Ni 0.75% La on New Norit ROX 0.8 | PNNL 59894-75-1 | PDLE 2 | 30 | 12.6 | 1.5 | 290/12 h | 100% |
| F180 | 5% Ni + 0.54% Pr + 0.22% Ce on 570393 | PNNL 59894-78-1 | PDLE 1 | 30 | 11.8 | 1.5 | 290/3 h | 100% |
| | Norit ROX 0.8 | | | | | | | |
| F181 | 5% Ni + 0.251% Pr + 2.249% Ce on | PNNL 59894-81-1 | PDLE 2 | 30 | 13.69 | 1.5 | 290/3 h | 100% |
| | 570393 Norit ROX 0.8 | | | | | | | |

Approximately 30 cc of catalyst was loaded into the reactor. Reduction of the catalyst was conducted at 290° C. under 250 sccm of pure H₂ flow for approximately 3 hours. The catalyst was tested using a trickle bed reactor with reagent grade glycerol (Fisher) as feed. Approximately 1% sodium hydroxide (NaOH) base was added to the feedstock solution. Reaction temperatures ranged from 180-210° C., and reaction pressure ranged from 1200-1600 psig. Liquid feed rates ranged from 35-50 mL/h, with H₂ flows from approximately 318 to 454 sccm. The product recovery vessel collected product solution at atmospheric pressure and at sub-ambient temperatures. A chiller unit used to cool the product collection vessels was not used, but if used it would likely aid in the greater capture of product. Some of the volatiles were most likely lost resulting in lower carbon recoveries. The test results are summarized in Table 4 below.

TABLE 4
Modified Nickel/Praseodymium/Cerium (F181) performance

| System Conditions | | | | | | | |
|---|---|---|---|---|---|---|---|
| 5% Ni + 0.251% Pr + 2.249% Ce on 570393 Norit | F181-1 | F181-2G | F181-3 | F181-4 | F181-5G | F181-6 | F181-7 |
| Total hours on stream | 268.10 | 289.50 | 317.08 | 339:26:00 | 364:45:00 | 460.50 | 601.78 |
| Cat. Bed Temp (° C.) | 191 | 210 | 180 | 210 | 190 | 191 | 210 |
| System Pressure (psi) | 1200 | 1200 | 1200 | 1200 | 1600 | 1600 | 1600 |
| Liq. Feed Rate (ml/hr) | 35 | 35 | 35 | 35 | 35 | 35 | 35 |
| Feed Concentration (wt %) | 39.63 | 39.63 | 20.40 | 20.40 | 20.40 | 39.63 | 39.63 |
| Source (Glycerol/Xylitol) | Glycerol | Glycerol | Xylitol | Xylitol | Xylitol | Glycerol | Glycerol |
| NaOH Feed Concentration (wt %) | 1.00 | 1.00 | 1.00 | 1.00 | 1.00 | 1.00 | 1.00 |
| H2 Flow Rate (SCCM) | 318 | 318 | 318 | 318 | 318 | 318 | 318 |
| % Wt. Recovery | 93.21 | | 84.19 | 88.90 | | 91.17 | 95.67 |
| % Carbon Recovery | 86.69 | | 73.89 | 89.77 | | 83.47 | 99.19 |
| Liquid Hourty Space Velocity (cc feed/cc cat/h) | 1.17 | 1.17 | 1.17 | 1.17 | 1.17 | 1.17 | 1.17 |
| Conversion (By Difference) | 68.1% | 80.7% | 92.6% | 96.7% | 95.5% | 49.8% | 78.0% |

Selectivites (carbon moter selectivity, normalized on total products recovered)

| | | | | | | | |
|---|---|---|---|---|---|---|---|
| PG C Molar Selectivity | 87.78% | Spot | 45.11% | 44.25% | Spot | 89.29% | 82.30% |
| EG C Molar Selectivity | 4.67% | Sample, | 28.55% | 29.14% | Sample, | 6.38% | 5.71% |
| Glycerol selectivity | 0.00% | conversion | 10.43% | 3.57% | conversion | 0.00% | 0.00% |
| Other Identified Polyols/Suqare Selectivity | 1.45% | only | 4.42% | 11.86% | only | 0.83% | 6.60% |
| Total Carboxylic selectivity | 4.24% | | 4.81% | 4.23% | | 3.51% | 3.44% |
| Methanol C Molar Selectivity | 0.05% | | 1.84% | 0.88% | | 0.00% | 0.04% |
| Ethanol C Molar Selectivity | 1.31% | | 0.00% | 1.94% | | 0.00% | 0.30% |
| 1-Propanol C Molar Selectivity | 0.00% | | 0.21% | 2.96% | | 0.00% | 0.03% |
| 2-Propanol C Molar Selectivity | 0.49% | | 2.32% | 0.00% | | 0.00% | 0.82% |
| Total mono Alcohol selectivity | 1.85% | | 4.37% | 5.78% | | 0.00% | 1.20% |
| Methane C Selectivity | 0.00% | | 0.00% | 0.00% | | 0.00% | 0.00% |

A “spot sample” means a check sample where a representative effluent sample is taken and the total concentration of the substrate in the product is compared against the total concentration of the substrate in the feed to generate an estimate of total conversion within a few percent. For a recovery sample, effluent is collected for a specific interval, usually 2 or more hours, and then the effluent sample is weighed and analyzed. The weight of substrate in the effluent is compared against the known weight of substrate fed to the reactor to generate conversion. The selectivity data is calculated by a normalized carbon molar selectivity. The total weight of each product in the effluent sample is converted into moles of carbon present as that product. This number is divided by the total moles of carbon consumed as substrate. Finally, this number is normalized by the total moles of carbon present from all detected products.

The results were surprising and unexpected. The glycerol conversion was around 50% with PG selectivity at 89%, nearly the baseline performance for Ni/Re. While these results are not as high as what is normally observed with an optimized rhenium-containing catalyst, this composition has not yet been optimized for this reaction. Pushing this catalyst resulted in higher conversions, but the selectivity suffered at higher temperatures even when the hydrogen pressure was adjusted to 1600 psi in order to increase hydrogen access to the catalyst at higher temperatures.

This catalyst resulted in 93% adjusted conversion of xylitol under the baseline conditions. It also had carbon molar selectivity to desired products of 45% to PG, 29% to ethylene glycol (EG), and 10% to glycerol. As it is a C5 compound, perfect selectivity for xylitol would be 1 mole of C3 and 1 mole of C2 per mole of xylitol. Thus, the theoretical maximum selectivity to PG would be 60% and the theoretical maximum for a perfect split would be 40% of the carbon going to EG.

Also for this run, we achieved 92% theoretical selectivity to desired C3 (PG+glycerol) and 72% theoretical selectivity to C2 (EG). Separating out the PG alone, we achieved 75% of theoretical selectivity just to PG. This passed the project milestone (of 75% of theoretical) and appears to be a good catalyst for both glycerol and xylitol conversion. This is a surprising result for the Ni/Pr/Ce modified catalyst. This catalyst composition has not been optimized, and further optimization research is definitely warranted.

Furthermore, this catalyst is made out of comparatively inexpensive metals. The estimated manufacturing costs of the Ni/Pr/Ce catalyst as well as the other catalysts disclosed herein are much lower than the rhenium-containing baseline catalysts, such as the Co/Pd/Re and Ni/Re formulations. And, importantly, the disclosed catalysts could render the metal loss issue presented with rhenium-containing catalysts mostly irrelevant. For instance, the Ni/Pr/Ce catalyst is prepared from rare earth metals that have almost no recovery value from the spent catalyst. If, as expected, this catalyst and those disclosed herein demonstrate similar activity to the baseline rhenium-containing catalysts, then metals loss would not significantly affect the recovery value, if at all. In fact, for the Ni/Pr/Ce catalyst along with those disclosed herein, the manufacturing costs are greater than the costs of the metals.

As an additional check, a plate was constructed on the combinatorial system in order to re-test a number of the catalysts as well as lab prepared materials. One purpose was to verify the Ni/La and Ni/Pr/Ce catalysts, as well as to test the reactivity of some of the supports used in prior tests. Catalysts were prepared at full impregnation. The reaction was performed at the usual conditions of 4 hours at 180° C. and 1400 psig, using a 10 wt % glycerol/1% NaOH feedstock.

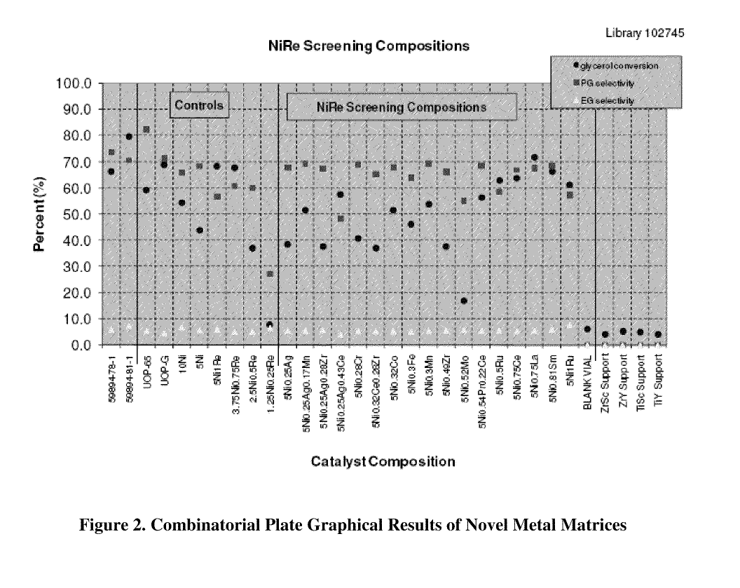

The graphical results are shown in FIG. 2 and in detailed form in Table 5.

TABLE 5
Combinatorial Plate Detailed Results of Novel Metal Matrices

| Catalyst | Glycerol conversion | PG selectivity | EG selectivity | Mass Recovery | Molar Balance |
|---|---|---|---|---|---|
| [symbol] -78-1 | 55.3 ± 1.3 | 73. [symbol] ± 1.4 | [symbol] .0 ± 0.2 | 92.9 ± 0.4 | 29.2 ± 0.9 |
| [symbol] -51-1 | 79.7 ± 1.1 | 70.5 ± 0.5 | 7.4 ± 0.0 | 92.4 ± 1.0 | 26.2 ± 0.6 |
| UOP-65 | 59.1 ± 0.3 | [symbol] ± 2.5 | 5.5 ± 0.0 | 19.7 ± 1.1 | 97.5 ± 1.6 |
| UOP- [symbol] | 63. [symbol] ± 4.3 | 71.3 ± 0.8 | 1.6 ± 0.1 | 93.0 ± 0.4 | 90.5 ± 1.1 |
| 1O [symbol] | 54.4 ± 4.4 | 66.0 ± 0.8 | 6.8 ± 0.3 | 91.9 ± 0.1 | 29.3 ± 0.6 |
| [symbol] | [symbol] ± 4.2 | 6 [symbol] .4 ± 1.6 | 5.7 ± 0.1 | 92.4 ± 0.0 | 90.9 ± 0.9 |
| [symbol] | 6 [symbol] .2 ± 0.9 | 56.7 ± 3.6 | 6.0 ± 0.1 | 91.7 ± 0.2 | 79.9 ± 7.8 |
| 1.7 [symbol] .75Re | 67.8 ± 0.8 | 60.7 ± 1.6 | 5.1 ± 0.2 | 92.3 ± 0.0 | 83.3 ± 1.1 |
| 2. [symbol] .5Re | 37.1 ± 3.3 | 60.1 ± 1.1 | 5.2 ± 0.2 | 92.4 ± 0.1 | 89.9 ± 1.2 |
| 1.2 [symbol] 25Re | 7.9 ± 1.1 | [symbol] .3 ± 3.6 | 6.6 ± 1.4 | 92.7 ± 0.1 | 95.2 ± 1.2 |
| [symbol] 25Ag | 38.2 ± 4.7 | 6 [symbol] .8 ± 1.1 | 5.6 ± 0.3 | 92.3 ± 0.3 | 91.5 ± 0.7 |
| [symbol] 25Ag0.17Mn | 51.5 ± 1.3 | [symbol] .3 ± 1.6 | 5.5 ± 0.2 | 92.2 ± 0.1 | 90.4 ± 0.7 |
| [symbol] 25Ag0.23Zr | 37.5 ± 1.2 | [symbol] .5 ± 0.4 | 5.1 ± 0.2 | 92.4 ± 0.0 | 91.5 ± 0.5 |
| [symbol] 25Ag0.42Ce | 57.5 ± 1.2 | [symbol] .3 ± 41.9 | 4.1 ± 3.5 | 92.3 ± 0.1 | 75.3 ± 23.2 |
| [symbol] 25Cr | 40.7 ± 2.9 | [symbol] .3 ± 1.9 | 5.4 ± 0.1 | 92.3 ± 0.2 | 91.5 ± 0.3 |
| [symbol] Co0.23Zr | 37.0 ± 2.3 | [symbol] .1 ± 2.2 | 5.4 ± 0.1 | 92.1 ± 0.4 | 91.0 ± 0.6 |
| [symbol] 2Co | 51.5 ± 1.3 | [symbol] .4 ± 1.7 | 5.4 ± 0.2 | 92.1 ± 0.1 | 89.2 ± 0.4 |
| [symbol] 2Fe | 46.3 ± 1.7 | [symbol] .6 ± 0.9 | 5.2 ± 0.1 | 91.4 ± 0.3 | 88.5 ± 0.5 |
| [symbol] 2Mn | 53.8 ± 1.5 | [symbol] .0 ± 1.7 | 5.5 ± 0.1 | 92.4 ± 0.1 | 90.8 ± 1.5 |
| [symbol] Zr | 37.5 ± 7.6 | [symbol] .2 ± 0.9 | 6.5 ± 0.6 | 92.1 ± 0.1 | 91.7 ± 0.6 |
| [symbol] Mo | 16.5 ± 4.4 | [symbol] ± 6.4 | 5.9 ± 0.1 | 92.1 ± 0.1 | 94.2 ± 0.1 |
| [symbol] Pro 22Ce | 56.5 ± 3.6 | [symbol] ± 1.0 | 5.5 ± 0.0 | 92.3 ± 0.2 | 88.6 ± 0.5 |
| [symbol] 6Ru | 62.9 ± 1.0 | [symbol] .5 ± 1.1 | 5.6 ± 0.0 | 92.5 ± 0.2 | 87.7 ± 0.3 |
| [symbol] 76Ce | 63.7 ± 2.1 | 58.7 ± 1.2 | 5.4 ± 0.1 | 91.7 ± 0.3 | 86.6 ± 0.6 |
| [symbol] 75La | 71.7 ± 1.4 | 67.0 ± 0.5 | 5.6 ± 0.2 | 92.7 ± 0.0 | 85.5 ± 0.4 |
| [symbol] Re | 66.3 ± 2.6 | 67.5 ± 0.2 | 5.9 ± 0.1 | 92.4 ± 0.1 | 87.9 ± 0.3 |
| [symbol] | 61.1 ± 1.5 | 57.4 ± 0.2 | 7.9 ± 0.3 | 92.2 ± 0.1 | 88.8 ± 0.5 |
| ELANK VIAl | 6.5 ± 0.4 | 0.0 ± 0.0 | 0.0 ± 0.0 | 93.1 ± 0.1 | 94.3 ± 0.4 |
| Zr [symbol] support | [symbol] .1 ± 0.4 | 0.0 ± 0.0 | 0.0 ± 0.0 | 92.5 ± 0.0 | 56.2 ± 0.4 |
| ZrY support | 5.2 ± 0.4 | 0.0 ± 0.0 | 0.0 ± 0.0 | 92.5 ± 0.1 | 55.2 ± 0.5 |
| Ti [symbol] support | 1.5 ± 0.2 | 0.0 ± 0.0 | 0.0 ± 0.0 | 92.5 ± 0.0 | 95.3 ± 0.2 |
| TiY support | 1.1 ± 0.3 | 0.0 ± 0.0 | 0.0 ± 0.0 | 93.1 ± 0.1 | 56.2 ± 0.3 |

[symbol]

indicates data missing or illegible when filed

In looking at FIG. 2, it is apparent that the disclosed embodiments are consistently comparable to the performance of the baseline catalysts, including rhenium-containing multimetallic catalysts. The Ni/La catalyst (8^{th} result from the right of the graph) provided for 72% glycerol conversion and 68% PG selectivity, which is similar to the UOP-G (Ni/Re) catalyst (4^{th} result from the left) at 69% glycerol conversion and 71% PG selectivity. (The composition of UOP-G is 5% Ni/1% Re on Norit® ROX 0.8 mm carbon extrudate. The composition of UOP-65 is 2.5% Co/0.45% Pd/2.4% Re on Norit® ROX 0.8 mm carbon extrudate.) The 5.0% Ni/0.54% Pr/0.22% Ce catalyst (11^{th} from the right) provided for 57% glycerol conversion and PG selectivity of 69%. However, an interesting point was that the 5.0% Ni/0.54% Pr/0.22% Ce catalysts (with modified and un-modified supports) demonstrated excellent results in combinatorial testing. The Ni/Pr/Ce catalyst (first on the left as 59894-78-1) demonstrated 67% glycerol conversion and 74% PG selectivity, while the modified Ni/Pr/Ce catalyst (second on the left as 59894-81-1) provided 80% glycerol conversion and 71% PG selectivity. Both of these catalysts beat the performance of the baseline catalyst.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A composition of matter comprising:
a solid multimetallic catalyst comprising nickel and at least one of La, Sm, Ce, Ru, Ag, Pr, Mn, Co, Pd, Cr, Mo, Zr, and Fe;
hydrogen; and
at least one of a carbon sugar, a carbon sugar alcohol, or glycerol.

2. The composition of claim 1 wherein the solid catalyst comprises a carbon support.

3. The composition of claim 2 wherein the solid catalyst comprises an acid washed extruded carbon support.

4. The composition of claim 1 wherein the multimetallic catalyst comprises nickel and lanthanum.

5. The composition of claim 1 wherein the multimetallic catalyst comprises nickel, praseodymium, and cerium.

6. The composition of claim 2 wherein the carbon support is modified with at least one of Zirconium Scandium (ZrSc), Zirconium Yttrium (ZrY), Titanium Scandium (TiSc), or Titanium Yttrium (TiY).

7. The composition of claim 3 wherein the carbon support is modified with at least one of Zirconium Scandium (ZrSc), Zirconium Yttrium (ZrY), Titanium Scandium (TiSc), or Titanium Yttrium (TiY).

8. The composition of claim 6 wherein the multimetallic catalyst comprises nickel and lanthanum.

9. The composition of claim 6 wherein the multimetallic catalyst comprises nickel, praseodymium, and cerium.

10. A composition of matter comprising:
a solid multimetallic catalyst comprising cobalt and at least one of Ni, Ir, Mo or Ce;
hydrogen; and
at least one of a carbon sugar, a carbon sugar alcohol, or glycerol.

11. The composition of claim 10 wherein the solid catalyst comprises a carbon support.

12. The composition of claim 11 wherein the solid catalyst comprises an acid washed extruded carbon support.

13. The composition of claim 11 wherein the carbon support is modified with at least one of Zirconium Scandium (ZrSc), Zirconium Yttrium (ZrY), Titanium Scandium (TiSc), or Titanium Yttrium (TiY).

14. The composition of claim 12 wherein the carbon support is modified with at least one of Zirconium Scandium (ZrSc), Zirconium Yttrium (ZrY), Titanium Scandium (TiSc), or Titanium Yttrium (TiY).

15. A solid multimetallic catalyst composition for the hydrogenolysis of glycerol for conversion to propylene glycol, comprising:
nickel and at least one of La, Sm, Ce, Ru, Ag, Pr, Mn, Co, Pd, Cr, Mo, Zr, and Fe.

16. The catalyst composition of claim 15 further comprising a carbon support.

17. The catalyst composition of claim 16 wherein the carbon support is an acid washed extruded carbon support.

18. The catalyst composition of claim 16 wherein the composition catalyst comprises nickel and lanthanum.

19. The catalyst composition of claim 16 wherein the composition catalyst comprises nickel, praseodymium, and cerium.

20. The catalyst composition of claim 16 wherein the carbon support is modified with at least one of Zirconium Scandium (ZrSc), Zirconium Yttrium (ZrY), Titanium Scandium (TiSc), or Titanium Yttrium (TiY).

21. The catalyst composition of claim 17 wherein the carbon support is modified with at least one of Zirconium Scandium (ZrSc), Zirconium Yttrium (ZrY), Titanium Scandium (TiSc), or Titanium Yttrium (TiY).

22. The catalyst composition of claim 20 wherein the catalyst comprises nickel and lanthanum.

23. The catalyst composition of claim 20 wherein the catalyst comprises nickel, praseodymium, and cerium.

24. A solid multimetallic catalyst composition for the hydrogenolysis of glycerol for conversion to propylene glycol, comprising:
cobalt and at least one of Ni, Ir, Mo or Ce.

25. The catalyst composition of claim 24 further comprising a carbon support.

26. The catalyst composition of claim 25 wherein the carbon support is an acid washed extruded carbon support.

27. The catalyst composition of claim 25 wherein the composition catalyst comprises cobalt/nickel.

28. The catalyst composition of claim 26 wherein the composition catalyst comprises cobalt/nickel.

29. The catalyst composition of claim 25 wherein the carbon support is modified with at least one of Zirconium Scandium (ZrSc), Zirconium Yttrium (ZrY), Titanium Scandium (TiSc), or Titanium Yttrium (TiY).

30. The catalyst composition of claim 26 wherein the carbon support is modified with at least one of Zirconium Scandium (ZrSc), Zirconium Yttrium (ZrY), Titanium Scandium (TiSc), or Titanium Yttrium (TiY).

31. The catalyst composition of claim 29 wherein the catalyst comprises cobalt/nickel.

32. The catalyst composition of claim 30 wherein the catalyst comprises cobalt/nickel.

33. A hydrogenolysis method comprising:
reacting a composition comprising a carbon sugar, a carbon sugar alcohol, or glycerol with hydrogen in the presence of a solid multimetallic catalyst comprising nickel and at least one of La, Sm, Ce, Ru, Ag, Pr, Mn, Co, Pd, Cr, Mo, Zr, and Fe.

34. The hydrogenolysis method of claim 33 wherein the catalyst further comprises a carbon support.

35. The hydrogenolysis method of claim 34 wherein the carbon support is an acid washed extruded carbon support.

36. The hydrogenolysis method of claim 34 wherein the carbon support is modified with at least one of Zirconium Scandium (ZrSc), Zirconium Yttrium (ZrY), Titanium Scandium (TiSc), or Titanium Yttrium (TiY).

37. A method of making propylene glycol comprising:
reacting a composition comprising glycerol with hydrogen in the presence of a solid multimetallic catalyst comprising nickel and at least one La, Sm, Ce, Ru, Ag, Pr, Mn, Co, Pd, Cr, Mo, Zr, and Fe.

38. The method of claim 37 wherein the catalyst further comprises a carbon support.

39. The method of claim 37 wherein the carbon support is an acid washed extruded carbon support.

40. The method of claim 38 wherein the carbon support is modified with at least one of Zirconium Scandium (ZrSc), Zirconium Yttrium (ZrY), Titanium Scandium (TiSc), or Titanium Yttrium (TiY).

41. The method of claim 37 wherein the multimetallic catalyst comprises nickel and lanthanum.

42. The method of claim 37 wherein the multimetallic catalyst comprises nickel, praseodymium, and cerium.

43. The method of claim 37 wherein the propylene glycol selectivity is in the range of 50% or greater.

44. The method of claim 37 wherein the reaction is carried out at a temperature from about 160° C. to about 240° C.

45. The method of claim 37 wherein reaction is carried out at a pressure from about 1200 to about 2200 psig.

46. A method of making propylene glycol comprising:
reacting a composition comprising glycerol with hydrogen in the presence of a solid multimetallic catalyst comprising cobalt and at least one of Ni, Ir, Mo or Ce.

47. The method of claim 46 wherein the catalyst further comprises a carbon support.

48. The method of claim 46 wherein the carbon support is an acid washed extruded carbon.

49. The method of claim 48 wherein the carbon support is modified with at least one of Zirconium Scandium (ZrSc), Zirconium Yttrium (ZrY), Titanium Scandium (TiSc), or Titanium Yttrium (TiY).

50. The method of claim 46 wherein the propylene glycol selectivity is in the range of 50% or greater.

51. The method of claim 46 wherein the reaction is carried out at a temperature from about 160° C. to about 240° C.

52. The method of claim 46 wherein reaction is carried out at a pressure from about 1200 to about 2200 psig.
